(19) United States
(12) Patent Application Publication
(10) Pub. No.: US 2023/0380775 A1
(43) Pub. Date: Nov. 30, 2023

(54) BIOLOGICAL INFORMATION ESTIMATING DEVICE AND BIOLOGICAL INFORMATION ESTIMATING METHOD

(71) Applicant: SHARP KABUSHIKI KAISHA, Sakai City (JP)

(72) Inventors: Azusa NAKANO, Sakai City (JP); Yoshitomo AKAGI, Sakai City (JP); Yoshihisa ADACHI, Sakai City (JP); Ryota TOMIZAWA, Sakai City (JP)

(21) Appl. No.: 18/202,904

(22) Filed: May 27, 2023

(30) Foreign Application Priority Data
May 30, 2022 (JP) 2022-087426
Mar. 17, 2023 (JP) 2023-042461

(51) Int. Cl. A61B 5/00; A61B 5/021; A61B 5/024

(52) U.S. Cl. CPC A61B 5/7278; A61B 5/024; A61B 5/02108; A61B 5/721

(57) ABSTRACT

A biological information estimating device includes: a detection unit configured to detect a pulse wave on a living organism; a modifying unit configured to determine whether or not the pulse wave contains disturbance and to modify a length of a measurement period based on a result of the determination; and an estimating unit configured to estimate biological information from the pulse wave detected in the measurement period.

### FIELD OF THE INVENTION

The present disclosure relates to biological information estimating devices and biological information estimating methods. The present application claims the benefit of priority to Japanese Patent Application, Tokugan, No. 2022-087426 filed on May 30, 2022 and to Japanese Patent Application, Tokugan, No. 2023-042461 filed on Mar. 17, 2023, the entire contents of which are incorporated herein by reference.

### BACKGROUND OF THE INVENTION

A cuff-type electronic blood pressure meter changes pressure inside the cuff In addition, the cuff itself serves as a sensor so that the cuff can detect a pulse wave. In addition, the blood pressure is estimated from the detected pulse wave.

Japanese Unexamined Patent Application Publication, Tokukai, No. 2015-16188 discloses a biological information processing device.

This biological information processing device detects biosignals including a pulse wave component and body motion noise components. In addition, the pulse wave component and the body motion noise components are separated. In addition, the separated pulse wave component is determined as a pulse wave signal (Abstract).

### SUMMARY OF THE INVENTION

Precision in estimating the blood pressure falls in cuff-type electronic blood pressure meters when the living organism the blood pressure of which is to be estimated has moved or when the living organism has made a vocal sound.

The biological information processing device disclosed in Japanese Unexamined Patent Application Publication, Tokukai, No. 2015-16188 needs time to change pressure inside the cuff and for this reason could take a relatively long time to detect a biosignal, possibly causing the living organism to waste time.

The present disclosure has been made in view of these issues. The present disclosure, in an aspect thereof, has an object to provide a biological information estimating device and a biological information estimating method both capable of, for example, increasing precision in estimating biological information and reducing the time taken to estimate the biological information.

The present disclosure, in an aspect thereof, is directed to a biological information estimating device including: a detection unit configured to detect a pulse wave on a living organism; a modifying unit configured to determine whether or not the pulse wave contains disturbance and to modify a length of a measurement period based on a result of the determination; and an estimating unit configured to estimate biological information from the pulse wave detected in the measurement period.

The present disclosure, in another aspect thereof, is directed to a biological information estimating method including: a) detecting a pulse wave on a living organism; b) determining whether or not the pulse wave contains disturbance and modifying a length of a measurement period based on a result of the determination; and c) estimating biological information from the pulse wave detected in the measurement period.

### BRIEF DESCRIPTION OF DRAWINGS

FIG. 1 is a block diagram of a biological information estimating device in accordance with Embodiment 1.

FIG. 2 is a diagram illustrating an example of disturbance the presence/absence of which is determined by a modifying unit included in the biological information estimating device in accordance with Embodiment 1.

FIG. 3 is a block diagram of an image processing unit included in the biological information estimating device in accordance with Embodiment 1.

FIG. 4 is a flow chart representing a flow of a process performed by the modifying unit included in the biological information estimating device in accordance with Embodiment 1.

FIG. 5 is a graph representing an exemplary timing for the modifying unit included in the biological information estimating device in accordance with Embodiment 1 to end a measurement period when the pulse wave contains no disturbance.

FIG. 6 is a graph representing an exemplary timing for the modifying unit included in the biological information estimating device in accordance with Embodiment 1 to end a measurement period when the pulse wave contains disturbance.

FIG. 7 is a graph representing a first example of local maximum point selection performed by the modifying unit included in the biological information estimating device in accordance with Embodiment 1.

FIG. 8 is a graph representing a first example of local minimum point selection performed by the modifying unit included in the biological information estimating device in accordance with Embodiment 1.

FIG. 9 is a graph representing a second example of local maximum point selection performed by the modifying unit included in the biological information estimating device in accordance with Embodiment 1.

FIG. 10 is a graph representing a second example of local minimum point selection performed by the modifying unit included in the biological information estimating device in accordance with Embodiment 1.

FIG. 11 is a graph representing an exemplary pulse waveform separated by the modifying unit included in the biological information estimating device in accordance with Embodiment 1.

FIG. 12 is a graph representing an exemplary first derivative of the pulse waveform separated by the modifying unit included in the biological information estimating device in accordance with Embodiment 1.

FIG. 13 is a graph representing an exemplary second derivative of the pulse waveform separated by the modifying unit included in the biological information estimating device in accordance with Embodiment 1.

FIG. 14 is a flow chart representing a flow of a process of determining the presence/absence of disturbance based on the pulse wave per se, the process being performed by the modifying unit included in the biological information estimating device in accordance with Embodiment 1.

FIG. 15 is a flow chart representing a flow of a process of determining the presence/absence of disturbance based on video, the process being performed by the modifying unit included in the biological information estimating device in accordance with Embodiment 1.

FIG. 16 is a flow chart representing a flow of a process of determining the presence/absence of disturbance based on sound, the process being performed by the modifying unit included in the biological information estimating device in accordance with Embodiment 1.

FIG. 17 is a flow chart representing a flow of a process of determining the presence/absence of disturbance based on either acceleration or angular velocity, the process being performed by the modifying unit included in the biological information estimating device in accordance with Embodiment 1.

### DESCRIPTION OF EMBODIMENTS

The following will describe an embodiment of the present disclosure with reference to drawings. Identical and equivalent elements in the drawings are denoted by the same reference numerals, and description thereof is not repeated.

### 1 Embodiment 1

### 1.1 Biological Information Estimating Device

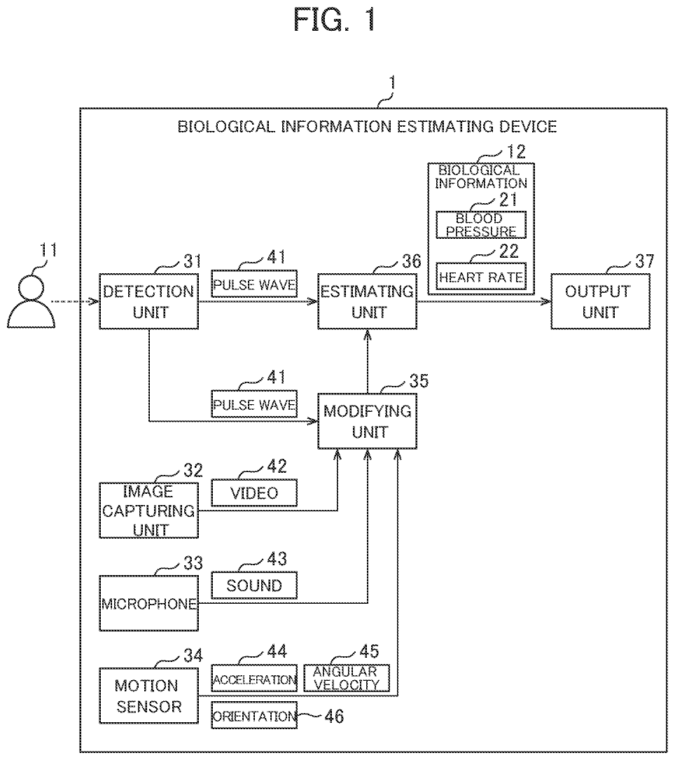

FIG. 1 is a block diagram of a biological information estimating device in accordance with Embodiment 1.

A biological information estimating device 1 shown in FIG. 1 estimates biological information 12 on a living organism 11. The living organism 11 on which the biological information 12 is estimated is a human. The living organism 11 may be a non-human animal. The estimated biological information 12 represents a condition of the living organism 11. The biological information 12 includes a blood pressure 21 and a pulse rate 22. The biological information 12 may include biological information other than the blood pressure 21 and the pulse rate 22. The pulse rate has the same meaning as heart rate in the present disclosure.

Referring to FIG. 1, the biological information estimating device 1 includes a detection unit 31, an image capturing unit 32, a microphone 33, a motion sensor 34, a modifying unit 35, an estimating unit 36, and an output unit 37.

The detection unit 31 detects a pulse wave 41 on the living organism 11.

The image capturing unit 32 captures images of the living organism 11 to obtain video 42. The image capturing unit 32 is built around, for example, an RGB camera.

The microphone 33 detects sound 43.

The motion sensor 34 detects acceleration 44, angular velocity 45, or orientation 46 of the detection unit 31. The motion sensor 34, when detecting the acceleration 44, includes an acceleration sensor that detects the acceleration 44. The motion sensor 34, when detecting the angular velocity 45, includes a gyrosensor that detects the angular velocity 45. The motion sensor 34, when detecting the orientation 46, includes an orientation sensor that detects the orientation 46.

The modifying unit 35 determines whether or not the pulse wave 41 contains disturbance and modifies the length of the measurement period on the basis of a result of the determination of the presence/absence of disturbance. This configuration enables adjusting the length of the measurement period suitably to the presence/absence of disturbance. The disturbance the presence/absence of which is determined is disturbance that reduces either periodicity or regularity of the pulse wave 41. The disturbance may be either external disturbance attributable to an event that occurs outside the system that produces the pulse wave 41 or internal disturbance attributable to an event that occurs inside the system. The modifying unit 35 may determine whether or not the pulse wave 41 contains disturbance either on the basis of the pulse wave 41 per se or on the basis of anything other than the pulse wave 41 including, for example, the video 42, the sound 43, the acceleration 44, the angular velocity 45, and the orientation 46. The modifying unit 35, upon having determined that the pulse wave 41 contains disturbance, increases the length of the measurement period. This configuration enables increasing the length of the measurement period to increase measurement precision of the biological information 12 when the pulse wave 41 contains such disturbance that precision in estimating the biological information 12 cannot increased unless the length of measurement period is increased. Meanwhile, the configuration enables reducing the length of the measurement period to reduce the time taken to estimate the biological information 12 when the pulse wave 41 contains no disturbance so that precision in estimating the biological information 12 can be increased even if the length of the measurement period is reduced. For example, the time taken to do so can be reduced to 10 seconds or even shorter.

The estimating unit 36 estimates the biological information 12 from the pulse wave 41 detected in the measurement period. The estimating unit 36 does not use, in estimating the biological information 12, the pulse wave 41 detected in a disturbance-present period that is a part of the measurement period and in which the pulse wave 41 is determined to contain disturbance. On the other hand, the estimating unit 36 uses, in estimating the biological information 12, the pulse wave 41 detected in a disturbance-absent period that is a part of the measurement period and in which the pulse wave 41 is determined to contain no disturbance.

The output unit 37 outputs the estimated biological information 12. The output unit 37 includes, for example, a display device that displays a screen showing the biological information 12, a speaker that projects sound representing the biological information 12, and/or a transmission circuit that transmits a signal representing the biological information 12.

### 1.2 Examples of Disturbance

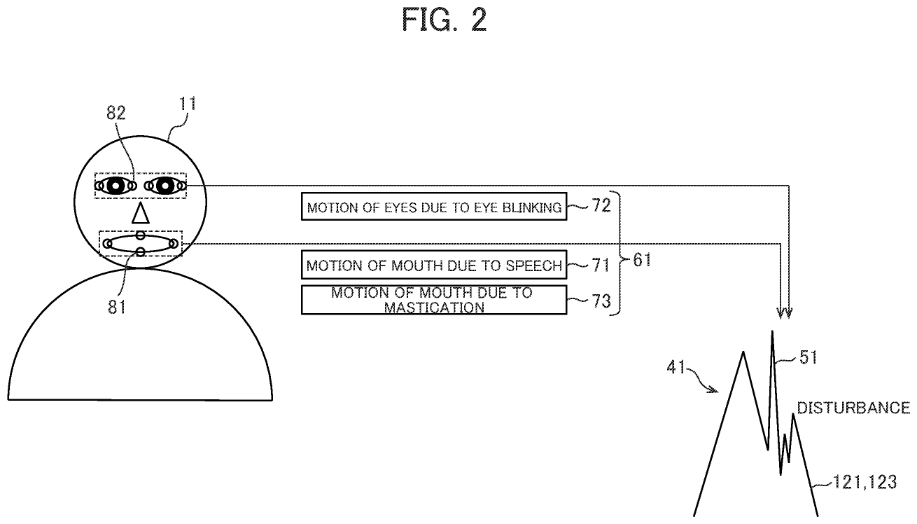

FIG. 2 is a diagram illustrating an example of disturbance the presence/absence of which is determined by the modifying unit included in the biological information estimating device in accordance with Embodiment 1.

Referring to FIG. 2, disturbance 51, the presence/absence of which on the pulse wave 41 is determined by the modifying unit 35, contains disturbance due to a motion 61 of the living organism 11. This configuration enables using, in estimating the biological information 12, the pulse wave 41 detected in the period in which the pulse wave 41 is determined to contain no disturbance 51 attributable to the motion 61 of the living organism 11. That in turn enables restraining precision from falling due to the motion 61 of the living organism 11 in estimating the biological information 12.

Referring to FIG. 2, the motion 61 of the living organism 11 includes, for example, a motion 71 of the mouth in speech, a motion 72 of the eyes in blinking, and a motion 73 of the mouth in mastication.

The modifying unit 35 detects a feature 81 of the mouth and detects, for example, the motion 71 of the mouth in speech and the motion 73 of the mouth in mastication on the basis of the detected feature 81 of the mouth. In addition, the modifying unit 35 detects a feature 82 of the eyes and detects, for example, the motion 72 of the eyes in blinking on the basis of the detected feature 82 of the eyes. Besides, the modifying unit 35 similarly detects, for example, the motion of the eyes, the motion of the mouth, and the motion of the eyebrow and cheek attributable to emotional changes as the motion 61 of the living organism 11 (not shown).

### 1.3 Detection Unit

The detection unit 31 detects the pulse wave 41 at a body part of the living organism 11. The body part where the pulse wave 41 is detected is the fingertip, the palm, the bottom of the foot, the cheek, the forehead, the nose, or the jaw. That body part may be any body part other than the fingertip, the palm, the bottom of the foot, the cheek, the forehead, the nose, and the jaw. The detection unit 31 may either detect the pulse wave 41 at one of these body parts or detect the pulse wave 41 simultaneously at some of the body parts. These plural body parts may be a combination of a fingertip of the left hand and a fingertip of the right hand, a combination of two or more body parts selected from the cheek, the forehead, and the nose, which are all parts of the face, or a combination of a fingertip and the face. The detection unit 31 preferably detects the pulse wave 41 in real time.

In a first example of detection of the pulse wave 41, the detection unit 31 includes a contact sensor.

The contact sensor is brought into contact with a fingertip of the living organism 11 to detect the pulse wave 41 at the fingertip that is in contact with the contact sensor.

The contact sensor includes a light-emitting unit and a light-receiving unit.

The light-emitting unit emits light. The emitted light may be either visible light and invisible light. The visible light is, for example, red light or green light. The invisible light is, for example, infrared light. The light-emitting unit includes, for example, a light-emitting diode (LED).

The light-receiving unit receives the light generated by the reflection and diffusion by the fingertip that is in contact with the detection unit 31 of the light emitted by the light-emitting unit and outputs a signal in accordance with the amount of the received light. Temporal changes of the amplitude of the outputted signal form the pulse wave 41 to be detected. This amount of light reflects the volume of the blood flowing in blood vessels inside the fingertip. The pulse wave 41 to be detected therefore reflects temporal changes in the volume of the blood. The light-receiving unit includes, for example, a photodiode.

The detection unit 31 may include a signal processing unit that performs signal processing on the detected pulse wave 41 to remove noise components from the pulse wave 41. The signal processing performed includes, for example, a process of removing long-period, low-frequency noise components and a process of removing short-period, high-frequency noise components. The former process is, for example, highpass filtering or trend removal. The latter process is, for example, lowpass filtering. The processing performed may be either an analog signal process implemented by an electronic circuit or a digital signal process implemented by a processor.

In a second example of detection of the pulse wave 41, the detection unit 31 includes an image capturing unit and an image processing unit.

The image capturing unit captures an image of the living organism 11 to obtain an image thereof. The image capturing unit includes, for example, an RGB camera. A single RGB camera may double as an image capturing unit included in the detection unit 31 and as the image capturing unit 32.

The image processing unit determines whether or not the obtained image contains the living organism 11, and upon determining that the image contains the living organism 11, outputs pixel values of the pixels at which the living organism 11 is imaged. Temporal changes of the outputted pixel values are the pulse wave 41 to be detected. The image processing unit includes, for example, a central processing unit (CPU) that executes programs. The processing performed by the image processing unit may be entirely or partially implemented by a dedicated electronic circuit.

When detecting the pulse wave 41 simultaneously at a fingertip of the left hand and at a fingertip of the right hand, the detection unit 31 includes a contact sensor for a left-hand fingertip and a contact sensor for a right-hand fingertip. The left-hand fingertip contact sensor is brought into contact with a fingertip of the left hand to detect the pulse wave 41 at the fingertip of the left hand that is in contact with the left-hand fingertip contact sensor. The right-hand fingertip contact sensor is brought into contact with a fingertip of the right hand to detect the pulse wave 41 at the fingertip of the right hand that is in contact with the right-hand fingertip contact sensor.

When the detection unit 31 detects the pulse wave 41 simultaneously at two or more body parts on the face, the image capturing unit captures an image of the face of the living organism 11 to obtain an image of the face. Additionally, the image processing unit computes the pulse wave 41 from the pixel values of the pixels at which the two or more body parts are imaged.

When the detection unit 31 detects the pulse wave 41 simultaneously at a fingertip and on the face, the contact sensor is brought into contact with the fingertip of the living organism 11 to detect the pulse wave 41 at the fingertip that is in contact with the contact sensor. Additionally, the image capturing unit captures an image of the face of the living organism 11 to obtain an image of the face. Additionally, the image processing unit computes the pulse wave 41 from the pixel values of the pixels at which the face is imaged.

### 1.4 Example of Image Processing Unit

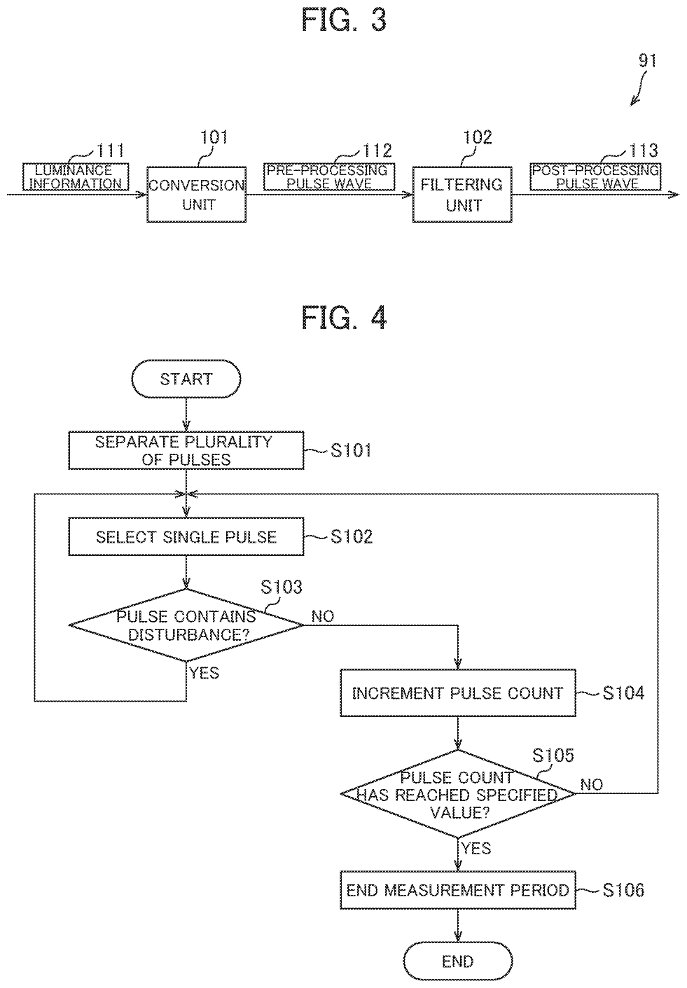

FIG. 3 is a block diagram of the image processing unit included in the biological information estimating device in accordance with Embodiment 1.

Referring to FIG. 3, an image processing unit 91 included in the detection unit 31 includes a conversion unit 101 and a filtering unit 102.

The conversion unit 101 converts, to a pre-processing pulse wave 112, luminance information 111 included in a region of interest (ROI) of the image obtained by the image capturing unit in the detection unit 31. The ROI is, for example, a region in which the cheek of the living organism 11 is imaged. The luminance information 111 is temporal changes of red, green, and blue luminance values in the ROI. The pre-processing pulse wave 112 is temporal changes of values representing the condition of the living organism 11. The conversion unit 101 converts red, green, and blue luminance values to those values by a conversion formula.

The filtering unit 102 performs bandpass filtering on the pre-processing pulse wave 112 to output a post-processing pulse wave 113. The bandpass filtering includes a process of removing high-frequency components such as flicker components from the pre-processing pulse wave 112 and a process of removing low-frequency components such as body motion components attributable to a gentle body motion including respiration from the pre-processing pulse wave 112.

### 1.5 Modifying Unit

The modifying unit 35 determines whether or not each pulse detected on the pulse wave 41 after the measurement period is started contains disturbance and ends the measurement period when the number of pulses, detected on the pulse wave 41 after the measurement period is started, that are determined to contain no disturbance has reached a specified value. The modifying unit 35 include, for example, a CPU for executing programs. The processing performed by the modifying unit 35 may be entirely or partially implemented by a dedicated electronic circuit.

The detected pulse wave 41 represents temporal changes of the detection amount detected on the living organism 11. Temporal changes of the pulse wave 41 contains cyclic temporal changes that result from cyclic heartbeats of the heart of the living organism 11. A pulse on the pulse wave 41 represents temporal changes of the detection amount, extracted from the pulse wave 41, in a period having a length of time taken by the heart of the living organism 11 to make one heartbeat. Both ends of the pulse may be either a local minimum point where the detection amount reaches a local minimum or a local maximum point where the detection amount reaches a local maximum. Therefore, both ends of the pulse may be either a trough or a peak on the waveform of the pulse wave 41.

Even if the number of pulses detected on the pulse wave 41 in the measurement period and determined to contain no disturbance 51 is equal to 1, the estimating unit 36 is still capable of estimating the biological information 12 from this single pulse. Therefore, the specified value may be equal to 1. However, the estimation of the biological information 12 by the estimating unit 36 becomes increasingly precise with a larger number of such pulses. Therefore, the specified value is preferably greater than or equal to 2 and more preferably greater than or equal to 5.

### 1.6 Processing Performed by Modifying Unit

FIG. 4 is a flow chart representing a flow of a process performed by the modifying unit included in the biological information estimating device in accordance with Embodiment 1.

The modifying unit 35 performs steps S101 to S106 shown in FIG. 4.

In step S101, the modifying unit 35 separates a plurality of pulses on the pulse wave 41.

In subsequent step S102, the modifying unit 35 selects one of the plurality of separated pulses that has not been selected so far.

In subsequent step S103, the modifying unit 35 determines whether or not the selected pulse contains the disturbance 51. If it is determined that the disturbance 51 is present, step S102 is performed again. If it is determined that the disturbance 51 is absent, step S104 is performed.

In step S104, the modifying unit 35 increments the pulse count by 1.

In steps S101 to S104, the modifying unit 35 retains the pulse count when the selected pulse is determined to contain the disturbance 51 and increments the pulse count when the selected pulse is determined to contain no disturbance 51. Therefore, the number of pulse counted by the modifying unit 35 is equal to the number of pulses determined to contain no disturbance 51.

In subsequent step S105, the modifying unit 35 determines whether or not the pulse count has reached the specified value. If it is determined that the pulse count has reached the specified value, step S106 is performed. If it is determined that the pulse count has not reached the specified value, again step S102 is performed.

In step S106, the modifying unit 35 ends the measurement period.

In steps S101 to S106, the modifying unit 35 determines whether or not each pulse on the pulse wave 41 contains the disturbance 51. If the number of pulses on the pulse wave 41 determined to contain no disturbance 51 has reached the specified value, the modifying unit 35 ends the measurement period.

### 1.7 Change in Length of Measurement Period in Response to Result of Determination of the Presence/Absence of Disturbance

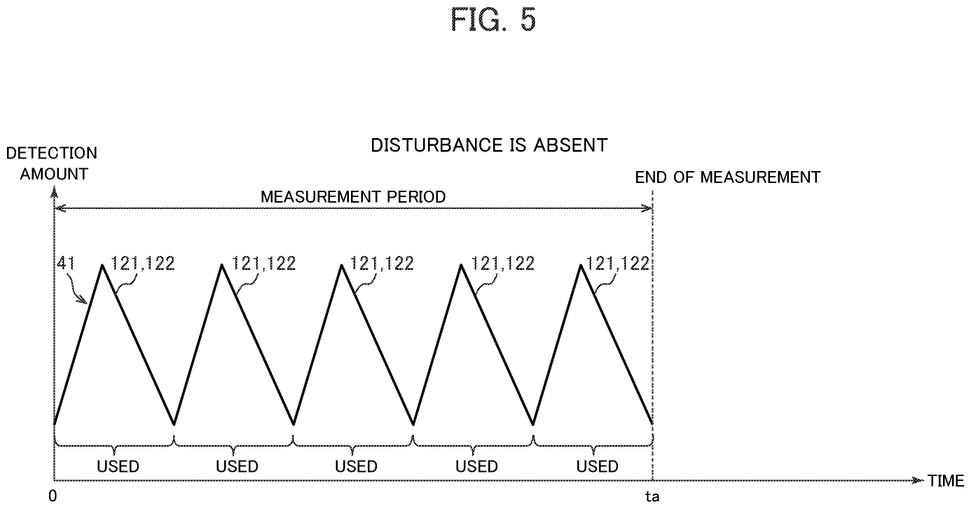

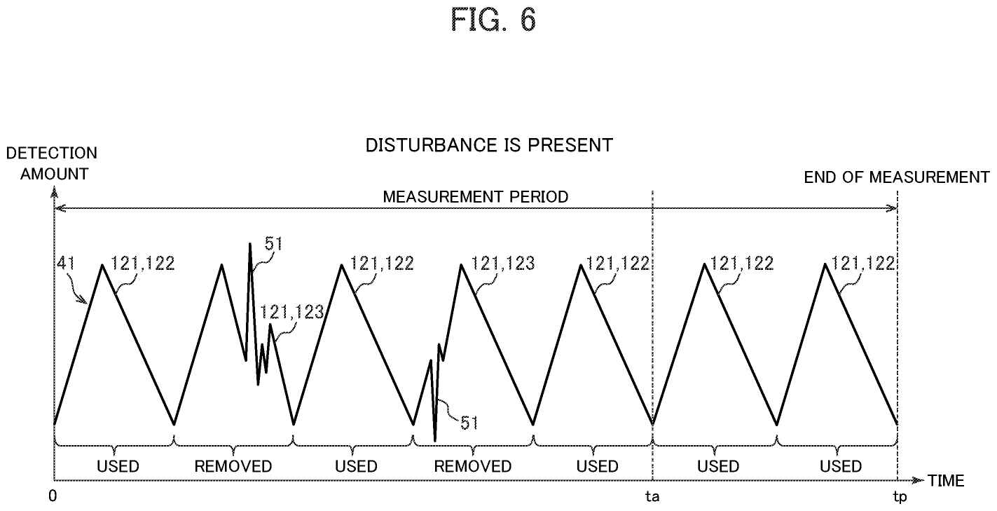

FIG. 5 is a graph representing an exemplary timing for the modifying unit included in the biological information estimating device in accordance with Embodiment 1 to end the measurement period when the pulse wave contains no disturbance. FIG. 6 is a graph representing an exemplary timing for the modifying unit included in the biological information estimating device in accordance with Embodiment 1 to end the measurement period when the pulse wave contains disturbance.

In FIGS. 5 and 6, time is plotted on the horizontal axis. In addition, the detection amount detected on the living organism 11 is plotted on the vertical axis.

In the examples shown in FIGS. 5 and 6, the modifying unit 35 determines whether or not each pulse 121 detected on the pulse wave 41 after the measurement period is started at time 0 contains the disturbance 51. The modifying unit 35 ends the measurement period when the number of pulses 122 on the pulse wave 41 that are determined to contain no disturbance 51 has reached 5. Therefore, the modifying unit 35 ends the measurement period at time ta, which is relatively early, as shown in FIG. 5, when the pulse wave 41 contains no disturbance 51. On the other hand, the modifying unit 35 ends the measurement period at time tp, which is relatively late, as shown in FIG. 6, when the pulse wave 41 contains the disturbance 51. The estimating unit 36 uses, in estimating the biological information 12, five of the pulses 122 determined to contain no disturbance 51 as shown in FIG. 5 when the pulse wave 41 contains no disturbance 51. On the other hand, the estimating unit 36 removes one or more pulses 123 determined to contain the disturbance 51, so that the pulses 123 are not used in estimating the biological information 12, and uses the five pulses 122 determined to contain no disturbance 51 in estimating the biological information 12 as shown in FIG. 6, when the pulse wave 41 contains the disturbance 51.

The modifying unit 35 separates the plurality of pulses 121 on the pulse wave 41 by performing signal processing on the pulse wave 41. The signal processing performed includes, for example, peak detection.

### 1.8 Separating Plurality of Pulses by Peak Detection

To separate the plurality of pulses 121 by performing peak detection on the pulse wave 41, the modifying unit 35 extracts local maximum points from the pulse wave 41 and specifies a portion of the pulse wave 41 that is present between adjacent local maximum points as one of the pulses 121. Alternatively, the modifying unit 35 extracts local minimum points from the pulse wave 41 and specifies a portion of the pulse wave 41 that is present between adjacent local minimum points as one of the pulses 121.

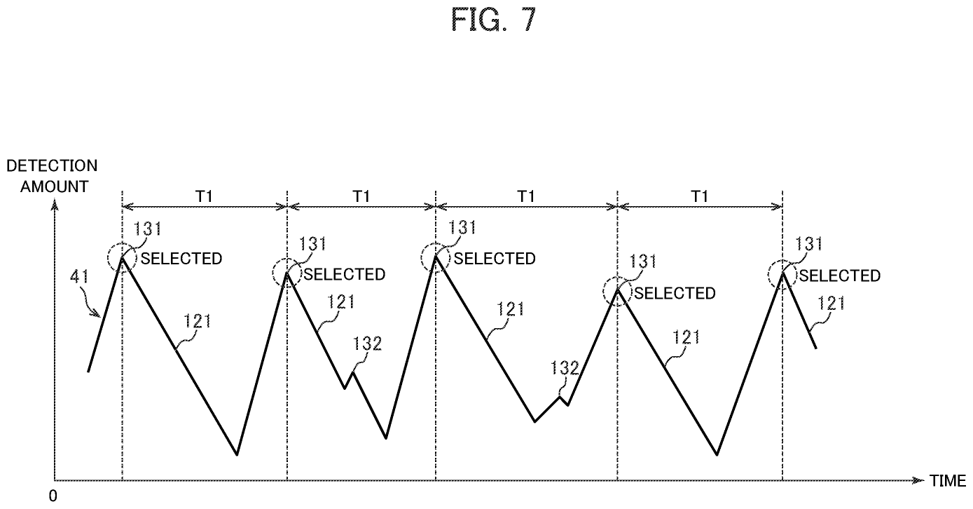

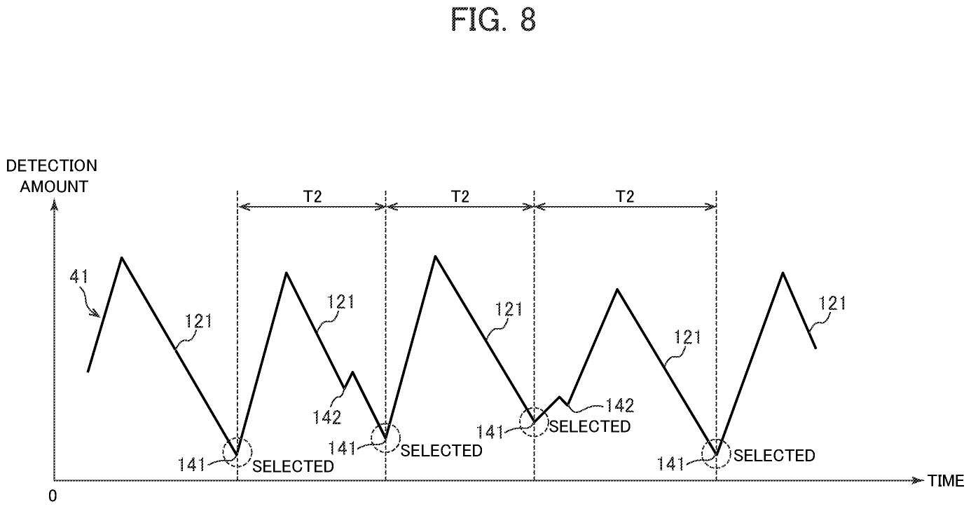

FIG. 7 is a graph representing a first example of local maximum point selection performed by the modifying unit included in the biological information estimating device in accordance with Embodiment 1. FIG. 8 is a graph representing a first example of local minimum point selection performed by the modifying unit included in the biological information estimating device in accordance with Embodiment 1.

In FIGS. 7 and 8, time is plotted on the horizontal axis. In addition, the detection amount detected on the living organism 11 is plotted on the vertical axis.

In the first example, the modifying unit 35 selects, in descending order of height, a plurality of local maximum points 131 detected on the pulse wave 41 after the start of the measurement period in such a manner that each time interval T1 between adjacent local maximum points 131 falls within a specified range, and specifies a portion of the pulse wave 41 that is present between adjacent local maximum points 131 as one of the pulses 121, as shown in FIG. 7. The range is specified on the basis of a typical pulse rate, which is 50 to 90 beats/minute. This configuration enables selecting overall local maximum points 131 that can reach serve as a suitable separation between two adjacent pulses 121. The configuration also enables restraining selection of regional local maximum points 132 that cannot serve as a suitable separation. Alternatively, as shown in FIG. 8, the modifying unit 35 selects, in descending order of depth, a plurality of local minimum points 141 detected on the pulse wave 41 after the start of the measurement period in such a manner that each time interval T2 between adjacent local minimum points 141 falls within a specified range, and specifies a portion of the pulse wave 41 that is present between adjacent local minimum points 141 as one of the pulses 121. The range is specified on the basis of a typical pulse rate, which is 50 to 90 beats/minute. This configuration enables selecting overall local minimum points 141 that can each serve as a suitable separation between two adjacent pulses 121. The configuration also enables restraining selection of regional local minimum points 142 that cannot serve as a suitable separation.

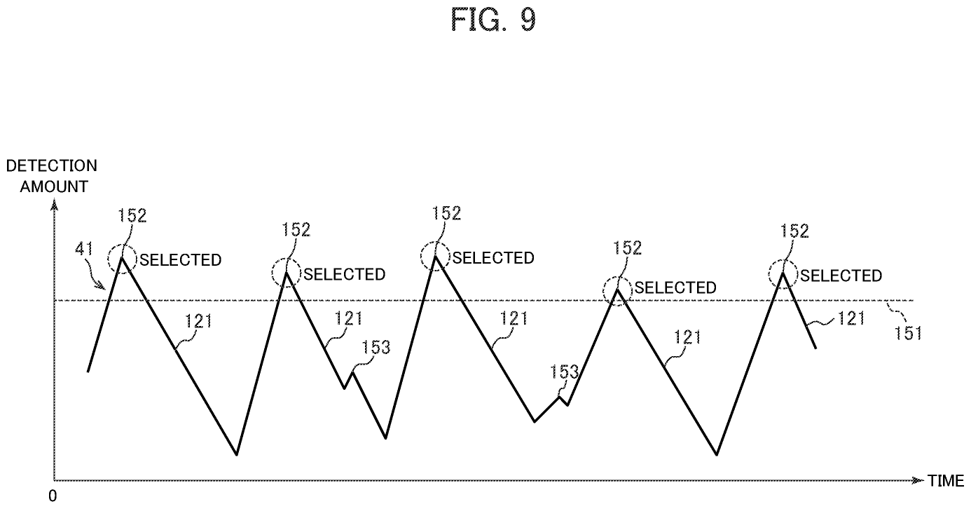

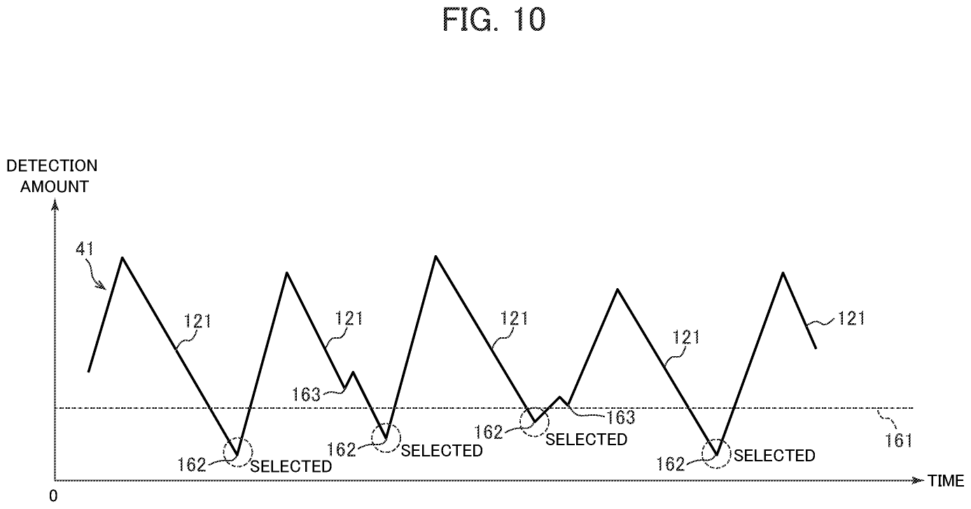

FIG. 9 is a graph representing a second example of local maximum point selection performed by the modifying unit included in the biological information estimating device in accordance with Embodiment 1. FIG. 10 is a graph representing a second example of local minimum point selection performed by the modifying unit included in the biological information estimating device in accordance with Embodiment 1.

In FIGS. 9 and 10, time is plotted on the horizontal axis. In addition, the detection amount detected on the living organism 11 is plotted on the vertical axis.

In the second example, the modifying unit 35 selects a plurality of local maximum points 152 that have a height greater than or equal to a height 151 specified based on the pulse wave 41 detected after the start of the measurement period and specifies a portion of the pulse wave 41 that is present between adjacent local maximum points 152 as one of the pulses 121, as shown in FIG. 9. The height 151 is specified on the basis of the amplitude of the pulse wave 41. This configuration enables selecting overall local maximum points 152 that can serve as a suitable separation between two adjacent pulses 121. The configuration also enables restraining selection of regional local maximum points 153 that cannot serve as a suitable separation. Alternatively, as shown in FIG. 10, the modifying unit 35 selects a plurality of local minimum points 162 that have a depth greater than or equal to a depth 161 specified based on the pulse wave 41 detected after the start of the measurement period and specifies a portion of the pulse wave 41 that is present between adjacent local minimum points 162 as one of the pulses 121. The depth 161 is specified on the basis of the amplitude of the pulse wave 41. This configuration enables selecting overall local minimum points 162 that can each serve as a suitable separation between two adjacent pulses 121. This configuration also enables restraining selection of regional local minimum points 163 that cannot serve as a suitable separation.

Alternatively, the local maximum points or the local minimum points may be selected using an SSF (slope sum function) filter that enhances the waveform.

### 1.9 Estimating Unit

The estimating unit 36 estimates the blood pressure 21 from the waveform and/or frequency dependence of the pulse wave 41 detected in a disturbance-absent period that is a part of the measurement period and that is determined to contain no disturbance 51. When the detection unit 31 detects the pulse wave 41 simultaneously at two or more body parts, the estimating unit 36 may estimate the blood pressure 21 from the correlation of the pulse waves 41 detected simultaneously at the two or more body parts. This configuration enables the biological information estimating device 1 to estimate the biological information 12 without a need to use a cuff.

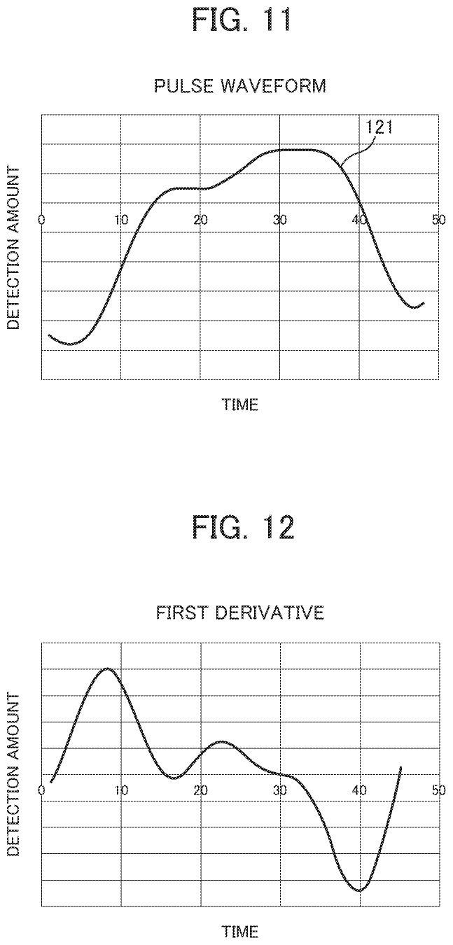

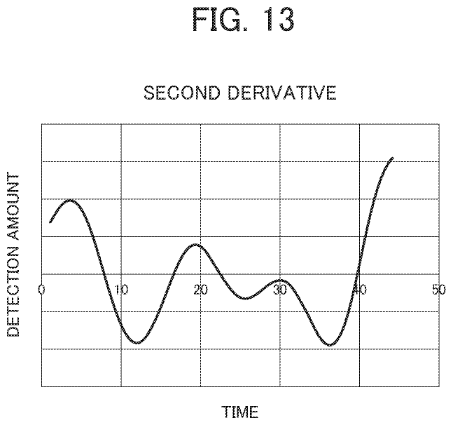

FIG. 11 is a graph representing an exemplary pulse waveform separated by the modifying unit included in the biological information estimating device in accordance with Embodiment 1. FIG. 12 is a graph representing an exemplary first derivative of the pulse waveform separated by the modifying unit included in the biological information estimating device in accordance with Embodiment 1. FIG. 13 is a graph representing an exemplary second derivative of the pulse waveform separated by the modifying unit included in the biological information estimating device in accordance with Embodiment 1.

In FIGS. 11, 12, and 13, time is plotted on the horizontal axis. In addition, the detection amount detected on the living organism 11 is plotted on the vertical axis.

The estimating unit 36 may measure the blood pressure 21 from the pulses 121 detected on the pulse wave 41 in a disturbance-absent period in the measurement period and determined to contain no disturbance 51. Here, the estimating unit 36 calculates a feature quantity from, for example, the waveform of the pulse 121 shown in FIG. 11, the first derivative thereof shown in FIG. 12, and the second derivative thereof shown in FIG. 13 and estimates the blood pressure 21 from the calculated feature quantity. The calculated feature quantity is, for example, an average value, a maximum value, a minimum value, a local maximum value, a local minimum value, the duration of the pulse 121, the value of the detection amount at the starting point or ending point of the pulse 121, a relationship between these values and threshold values, the number of local maximum values, and the number of local minimum values.

In addition, the estimating unit 36 estimates the pulse rate 22 from the number of the pulses 122 detected on the pulse wave 41 in a disturbance-absent period in the measurement period and determined to contain no disturbance 51. For example, the estimating unit 36 estimates the pulse rate 22 by dividing the number of the pulses 122 by the duration of the disturbance-absent period.

The estimating unit 36 is, for example, a CPU for executing programs. The processing performed by the modifying unit 35 may be entirely or partially implemented by a dedicated electronic circuit.

### 1.10 Determination of Presence/Absence of Disturbance Based on Pulse Wave Per Se

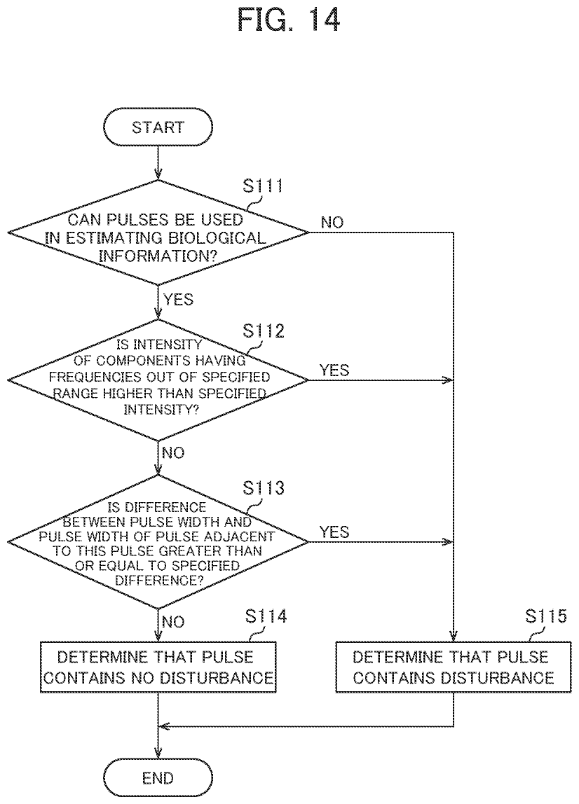

FIG. 14 is a flow chart representing a flow of a process of determining the presence/absence of disturbance based on the pulse wave per se, the process being performed by the modifying unit included in the biological information estimating device in accordance with Embodiment 1.

To determine whether or not the disturbance 51 is present on the pulses 121 on which a determination is to be made on the basis of the pulse wave 41 per se, the modifying unit 35 performs steps S111 to S115 shown in FIG. 14.

In step S111, the modifying unit 35 determines whether or not the pulses 121 can be used in estimating the biological information 12. For example, for the estimating unit 36 to calculate a feature quantity from each pulse 121 and estimate the blood pressure 21 from the calculated feature quantities, the modifying unit 35 determines that the pulses 121 cannot be used in estimating the biological information 12 when it is impossible to calculate, from the pulses 121, the feature quantity needed to estimate the blood pressure 21. The modifying unit 35 determines that the pulses 121 cannot be used in estimating the biological information 12, for example, when the number of local maximum values extractable from the first derivative shown in FIG. 12 is smaller than a reference, when the number of local minimum values extractable from the first derivative shown in FIG. 12 is smaller than a reference, when the number of local maximum values extractable from the second derivative shown in FIG. 13 is smaller than a reference, and when the number of local minimum values extractable from the second derivative shown in FIG. 13 is within a fixed range. If it is determined that the pulses 121 can be used in estimating the biological information 12, step S112 is performed. If it is determined that the pulses 121 cannot be used in estimating the biological information 12, step S115 is performed.

In step S112, the modifying unit 35 determines whether or not the intensity of the components of the pulses 121 that include frequencies out of the specified range is higher than a specified intensity. The modifying unit 35 performs frequency analysis on the pulses 121 to determine whether or not the intensity of the components is higher than a specified intensity. If it is determined that the intensity is higher than the specified intensity, step S115 is performed. If it is determined that the intensity is not higher than the specified intensity, step S113 is performed.

In step S113, the modifying unit 35 determines whether or not the difference between the pulse width of a pulse 121 and the pulse width of a pulse 121 adjacent to this pulse 121 is greater than or equal to a specified difference (threshold value). For example, when the former pulse width is less than or equal to 0.9 times the latter pulse width or when the former pulse width is greater than or equal to 1.1 times the latter pulse width, the modifying unit 35 determines that the difference between the former pulse width and the latter pulse width is greater than or equal to the specified difference. If it is determined that the difference is greater than or equal to the specified difference, step S115 is performed. If it is determined that the difference is not greater than or equal to the specified difference, step S114 is performed. Note that the aforementioned threshold value against which the difference between the pulse width of a pulse 121 and the pulse width of a pulse 121 adjacent to this pulse 121 is compared may be adjusted in accordance with the value of the estimated pulse rate. This is because the range of fluctuations of intervals between heart pulses can increase or decrease depending on the value of the pulse rate.

In step S114, the modifying unit 35 determines that the pulses 121 contain no disturbance 51.

In step S115, the modifying unit 35 determines that the pulses 121 contain the disturbance 51.

In steps S111 to S115, when the pulses 121 on which a determination is to be made cannot be used in estimating the biological information 12, the modifying unit 35 determines that the pulses 121 on which a determination is to be made contain the disturbance 51.

In addition, when the intensity of the components of the pulses 121 on which a determination is to be made and that include frequencies out of the specified range is higher than the specified intensity, the modifying unit 35 determines that the pulses 121 on which a determination is to be made contain the disturbance 51.

In addition, when the difference between the pulse width of a pulse 121 on which a determination is to be made and the pulse width of a pulse 121 adjacent to this pulse 121 on which a determination is to be made is greater than or equal to the specified difference, the modifying unit 35 determines that the pulse 121 on which a determination is to be made contains the disturbance 51. Hence, when the regularity or periodicity of the pulse wave 41 has deteriorated due to arrhythmia of the heart of the living organism 11, in other words, when the rhythm of the heartbeat becomes irregular, the modifying unit 35 determines that the pulse 121 on which a determination is to be made contains the disturbance 51. In addition, when the regularity or periodicity of the pulse wave 41 has deteriorated due to, for example, an error in the detection of the pulses 121, the modifying unit 35 determines that the pulse 121 on which a determination is to be made contains the disturbance 51.

### 1.11 Determination of Presence/Absence of Disturbance Based on Video

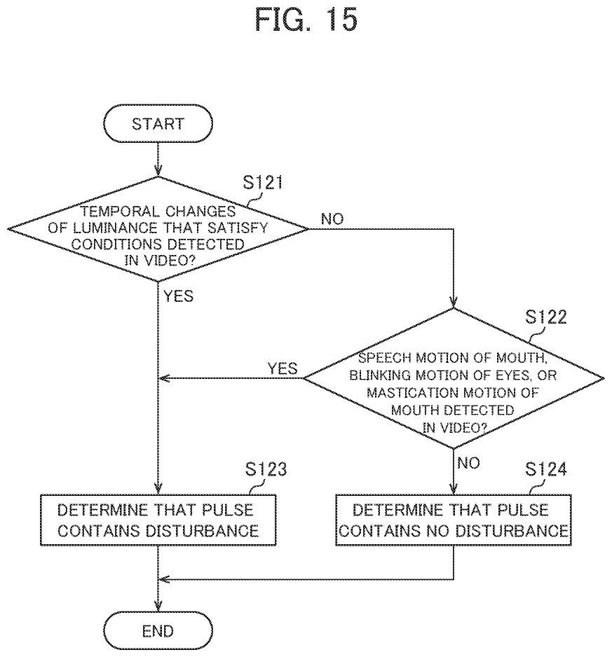

FIG. 15 is a flow chart representing a flow of a process of determining the presence/absence of disturbance based on video, the process being performed by the modifying unit included in the biological information estimating device in accordance with Embodiment 1.

To determine whether or not the disturbance 51 is present on the pulses 121 on which a determination is to be made on the basis of the video 42, the modifying unit 35 performs steps S121 to S124 shown in FIG. 15.

In step S121, the modifying unit 35 determines whether or not temporal changes of the luminance that satisfy conditions are detected in the video 42 in a period in which the pulse 121 is detected. The conditions to be satisfied include, for example, such conditions that changes per time of the luminance of the whole video 42 be greater than or equal to a threshold value. If it is determined that these temporal changes are detected in the video 42, step S123 is performed. If it is determined that the temporal changes are not detected in the video 42, step S122 is performed.

In step S122, the modifying unit 35 determines whether or not the motion 71 of the mouth due to speech, the motion 72 of the eyes due to eye blinking, or the motion 73 of the mouth due to mastication is detected in the video 42 in the period in which the pulse 121 is detected. If it is determined that the motion 71 of the mouth due to speech, the motion 72 of the eyes due to eye blinking, or the motion 73 of the mouth due to mastication is detected in the video 42, step S123 is performed. If it is determined that none of the motion 71 of the mouth due to speech, the motion 72 of the eyes due to eye blinking, and the motion 73 of the mouth due to mastication is detected in the video 42, step S124 is performed.

In step S123, the modifying unit 35 determines that the pulse 121 contains the disturbance 51.

In step S124, the modifying unit 35 determines that the pulse 121 contains no disturbance 51.

In steps S121 to S124, when temporal changes of the luminance that satisfy conditions are detected in the video 42 in a period in which the pulse 121 on which a determination is to be made is detected, the modifying unit 35 determines that the pulse 121 on which a determination is to be made contains the disturbance 51. In addition, when the motion 71 of the mouth due to speech, the motion 72 of the eyes due to eye blinking, or the motion 73 of the mouth due to mastication is detected in the video 42 in the period in which the pulse 121 on which a determination is to be made is detected, the modifying unit 35 determines that the pulse 121 on which a determination is to be made contains the disturbance 51. In other words, in step S121 of determining whether or not temporal changes of the luminance that satisfy conditions are detected in the video 42 in the period in which the pulse 121 is detected, a disturbance caused by a motion beyond expectation of the living organism side that is a subject side and/or a disturbance caused by a motion of the image capturing unit 32 that is an imaging side can be detected.

### 1.12 Determination of Presence/Absence of Disturbance Based on Sound

FIG. 16 is a flow chart representing a flow of a process of determining the presence/absence of disturbance based on sound, the process being performed by the modifying unit included in the biological information estimating device in accordance with Embodiment 1.

To determine whether or not the pulse 121 on which a determination is to be made contains disturbance on the basis of the sound 43, the modifying unit 35 performs steps S131 to S134 shown in FIG. 16.

In step S131, the modifying unit 35 determines whether or not the sound 43 with a sound volume greater than or equal to a specified sound volume is detected by the microphone 33 in a period in which the pulse 121 is detected. If it is determined that the sound 43 is detected, step S133 is performed. If it is determined that the sound 43 is not detected, step S132 is performed.

In step S132, the modifying unit 35 determines whether or not the sound 43 due to speech by the living organism 11 is detected by the microphone 33 in the period in which the pulse 121 is detected. The modifying unit 35 detects the sound 43 by an audio recognition function. If it is determined that the sound 43 is detected, step S133 is performed. If it is determined that the sound 43 is not detected, step S134 is performed.

In step S133, the modifying unit 35 determines that the pulse 121 contains the disturbance 51.

In step S134, the modifying unit 35 determines that the pulse 121 contains no disturbance 51.

In steps S131 to S134, when the sound 43 with a sound volume greater than or equal to the specified sound volume is detected by the microphone 33 in the period in which the pulse 121 on which a determination is to be made is detected, the modifying unit 35 determines that the pulse 121 on which a determination is to be made contains the disturbance 51. In addition, when sound due to speech by the living organism 11 is detected by the microphone 33 in the period in which the pulse 121 on which a determination is to be made is detected, the modifying unit 35 determines that the pulse 121 on which a determination is to be made contains the disturbance 51.

### 1.13 Determination of Presence/Absence of Disturbance Based on Acceleration or Angular Velocity

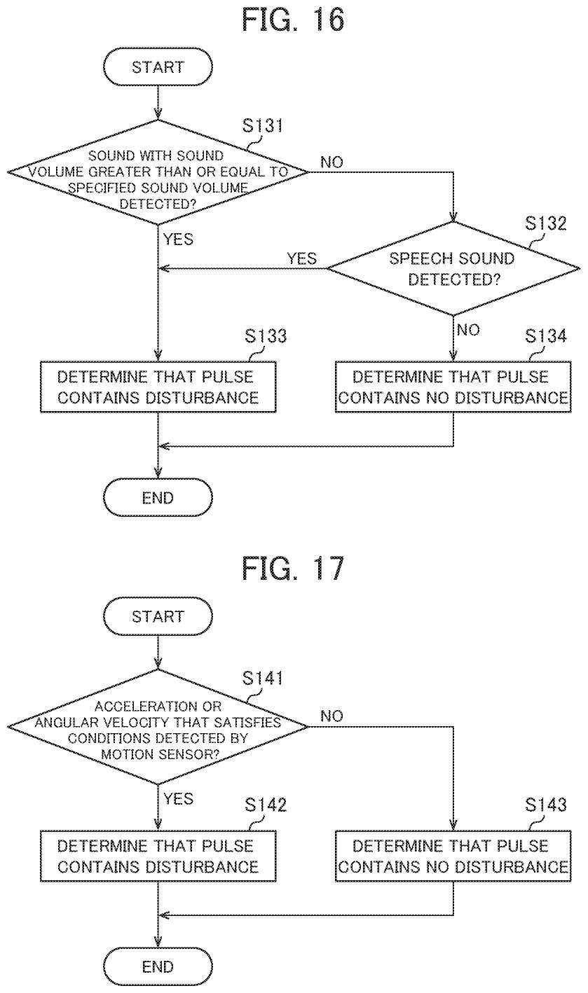

FIG. 17 is a flow chart representing a flow of a process of determining the presence/absence of disturbance based on either acceleration or angular velocity, the process being performed by the modifying unit included in the biological information estimating device in accordance with Embodiment 1.

To determine whether or not the disturbance 51 is present on the pulses 121 on which a determination is to be made on the basis of the acceleration 44 or the angular velocity 45, the modifying unit 35 performs steps S141 to S143 shown in FIG. 17.

In step S141, the modifying unit 35 determines whether or not either the acceleration 44 or the angular velocity 45 that satisfies conditions is detected by the motion sensor 34 in a period in which the pulses 121 are detected. The conditions to be satisfied include, for example, such conditions that either the acceleration 44 or the angular velocity 45 have a greater magnitude than a specified magnitude. If it is determined that either the acceleration 44 or the angular velocity 45 that satisfies conditions is detected, step S142 is performed. If it is determined that neither the acceleration 44 nor the angular velocity 45 that satisfies conditions is detected, step S143 is performed.

In step S142, the modifying unit 35 determines that the pulse 121 contains the disturbance 51.

In step S143, the modifying unit 35 determines that the pulse 121 contains no disturbance 51.

In steps S141 to S143, when either the acceleration 44 or the angular velocity 45 that satisfies conditions is detected by the motion sensor 34 in the period in which the pulse 121 on which a determination is to be made is detected, the modifying unit 35 determines that the pulse 121 on which a determination is to be made contains the disturbance 51. Hence, when the detection unit 31 has moved much, the modifying unit 35 determines that the pulse 121 on which a determination is to be made contains the disturbance 51.

Note that the same description applies to cases where the orientation 46 is detected by the motion sensor 34 (not shown). The motion sensor 34 is, when attached to the detection unit 31, capable of detecting the presence/absence of disturbance due to a motion of the detecting side. Alternatively, the motion sensor 34 may be provided on the living organism that is a subject side by being mounted to, for example, an accessory or a decorative object such as a ring on the finger, the necklace, the hat, or the glasses. When the motion sensor 34 is provided on the living organism, the detection unit 31 may, when a motion is detected that is greater than or equal to a prescribed threshold value, determine that there has been disturbance, by notifying to the detection unit via a wired or wireless link (not shown).

The present disclosure is not limited to the description of the embodiments and examples above. Any structure detailed in the embodiments and examples may be replaced by a practically identical structure, a structure that achieves the same effect and function, or a structure that achieves the same purpose.

What is claimed is:

1. A biological information estimating device comprising:
a detection unit configured to detect a pulse wave on a living organism;
a modifying unit configured to determine whether or not the pulse wave contains disturbance and to modify a length of a measurement period based on a result of the determination; and
an estimating unit configured to estimate biological information from the pulse wave detected in the measurement period.

2. The biological information estimating device according to claim 1, wherein the modifying unit increases the length when it is determined that the pulse wave contains the disturbance.

3. The biological information estimating device according to claim 1, wherein the disturbance includes disturbance caused by a motion of the living organism.

4. The biological information estimating device according to claim 1, wherein the disturbance includes disturbance caused by a motion of the living organism and/or disturbance caused by a motion of the biological information estimating device.

5. The biological information estimating device according to claim 1, wherein the modifying unit determines whether or not each pulse detected on the pulse wave after the measurement period is started contains the disturbance and, when a count of pulses detected on the pulse wave after the measurement period is started and determined not to contain the disturbance has reached a specified value, ends the measurement period.

6. The biological information estimating device according to claim 5, wherein when a pulse on which the determination is to be made cannot be used in estimating the biological information, the modifying unit determines that the pulse on which the determination is to be made contains the disturbance.

7. The biological information estimating device according to claim 5, wherein when a pulse on which the determination is to be made has a component that has a frequency out of a specified range and that has an intensity higher than a specified intensity, the modifying unit determines that the pulse on which the determination is to be made contains the disturbance.

8. The biological information estimating device according to claim 5, wherein when a difference between a pulse width of a pulse on which the determination is to be made and a pulse width of a pulse adjacent to this pulse on which the determination is to be made is greater than or equal to a specified difference, the modifying unit determines that the pulse on which the determination is to be made contains the disturbance.

9. The biological information estimating device according to claim 5, further comprising an image capturing unit configured to capture an image of the living organism to obtain video, wherein when a temporal change of luminance that satisfies a condition is detected in the video in a period in which a pulse on which the determination is to be made is detected, the modifying unit determines that the pulse on which the determination is to be made contains the disturbance.

10. The biological information estimating device according to claim 5, further comprising an image capturing unit configured to capture an image of the living organism to obtain video, wherein when a motion due to speech by the living organism is detected in the video in a period in which a pulse on which the determination is to be made is detected, the modifying unit determines that the pulse on which the determination is to be made contains the disturbance.

11. The biological information estimating device according to claim 1, wherein the biological information includes blood pressure.

12. The biological information estimating device according to claim 1, wherein the biological information includes a pulse rate.

13. A biological information estimating method comprising:
a) detecting a pulse wave on a living organism;
b) determining whether or not the pulse wave contains disturbance and modifying a length of a measurement period based on a result of the determination; and
c) estimating biological information from the pulse wave detected in the measurement period.

14. The biological information estimating device according to claim 2, wherein the disturbance includes disturbance caused by a motion of the living organism.

15. The biological information estimating device according to claim 2, wherein the disturbance includes disturbance caused by a motion of the living organism and/or disturbance caused by a motion of the biological information estimating device.

16. The biological information estimating device according to claim 2, wherein the modifying unit determines whether or not each pulse detected on the pulse wave after the measurement period is started contains the disturbance and, when a count of pulses detected on the pulse wave after the measurement period is started and determined not to contain the disturbance has reached a specified value, ends the measurement period.

17. The biological information estimating device according to claim 16, wherein when a pulse on which the determination is to be made cannot be used in estimating the biological information, the modifying unit determines that the pulse on which the determination is to be made contains the disturbance.

18. The biological information estimating device according to claim 16, wherein when a pulse on which the determination is to be made has a component that has a frequency out of a specified range and that has an intensity higher than a specified intensity, the modifying unit determines that the pulse on which the determination is to be made contains the disturbance.

19. The biological information estimating device according to claim 16, wherein when a difference between a pulse width of a pulse on which the determination is to be made and a pulse width of a pulse adjacent to this pulse on which the determination is to be made is greater than or equal to a specified difference, the modifying unit determines that the pulse on which the determination is to be made contains the disturbance.

20. The biological information estimating device according to claim 16, further comprising an image capturing unit configured to capture an image of the living organism to obtain video, wherein when a temporal change of luminance that satisfies a condition is detected in the video in a period in which a pulse on which the determination is to be made is detected, the modifying unit determines that the pulse on which the determination is to be made contains the disturbance.
